(19) United States
(12) Patent Application Publication
(10) Pub. No.: US 2026/0363709 A1
(43) Pub. Date: Nov. 19, 2026

(54) SURGICAL DEVICE FOR PIERCING A BONE

(71) Applicant: Dereck PEERY, ARDMORE, OK (US)

(72) Inventor: Dereck PEERY, ARDMORE, OK (US)

(21) Appl. No.: 19/630,152

(22) Filed: Mar. 26, 2026

Related U.S. Application Data
(60) Provisional application No. 63/805,550, filed on May 14, 2025.

(51) Int. Cl. A61B 17/16; A61B 17/34

(52) U.S. Cl. CPC A61B 17/1604; A61B 17/3472; A61B 2017/347

(57) ABSTRACT

A surgical device for piercing bone has a cannula. A trocar is positionable within the cannula. An inner elongated pin is positionable within the trocar. An actuation member is connected at an end of the inner elongated pin. The inner elongated pin is deployable and retractable. When in a deployed state, an inner elongated pin tip is positioned a greater distance out of a trocar terminating end than when in a retracted state. The inner elongated pin is deployable and retractable while positioned within the trocar.

### CROSS REFERENCE TO RELATED APPLICATION

This application claims benefit of U.S. Provisional Application Ser. No. 63/805,550 entitled, “Surgical Device for Piercing a Bone” filed May 14, 2025, the entire disclosure of which is incorporated herein by reference.

### FIELD OF THE DISCLOSURE

The present disclosure is generally related to surgical devices and more particularly is related to surgical devices for piercing a bone.

### BACKGROUND OF THE DISCLOSURE

Certain orthopedic surgeries require surgical hardware to be placed within a bone structure. In some procedures where delicate anatomy is involved, the use of hand tools, such as trocars, are preferred. A trocar is used to pierce the bone, whereafter surgical hardware can be positioned within the hole or opening created in the bone. Trocars used in such procedures require precise positioning on the bone prior to being struck by a mallet or hammer, which drives the trocar into the bone. However, current trocar systems often feature blunt tips and are prone to slippage when struck by a mallet or hammer, which can cause the tip of the trocar to move to an unintended or undesirable location on the bone. Thus, slow precision is typically required when using trocars in this manner, particularly on irregularly shaped or small bone structures which have a propensity to cause slippage. Positioning and re-positioning the trocar on the bone as a result of slippage may result in extended surgery times, a decrease in the quality of the surgery, or extended recovery time for the patient.

### SUMMARY OF THE DISCLOSURE

Embodiments of the present disclosure provide a system and method for a surgical device for piercing bone. Briefly described, in architecture, one embodiment of the system, among others, can be implemented as follows. A surgical device for piercing bone has a cannula. A trocar is positionable within the cannula. An inner elongated pin is positionable within the trocar. An actuation member is connected at an end of the inner elongated pin. The inner elongated pin is deployable and retractable. When in a deployed state, an inner elongated pin tip is positioned a greater distance out of a trocar terminating end than when in a retracted state. The inner elongated pin is deployable and retractable while positioned within the trocar.

The present disclosure can also be viewed as providing methods of piercing bone. In this regard, one embodiment of such a method, among others, can be broadly summarized by the following steps: positioning a trocar within a cannula, and positioning an inner elongated pin within the trocar, wherein an actuation member is connected at an end of the inner elongated pin, wherein the inner elongated pin is deployable and retractable, wherein when in a deployed state, a tip of the inner elongated pin is positioned a greater distance out of a terminating end of the trocar than when in a retracted state, and wherein the inner elongated pin is deployable and retractable while positioned within the trocar.

The present disclosure can also be viewed as providing an apparatus for piercing bone. Briefly described, in architecture, one embodiment of the apparatus, among others, can be implemented as follows. A surgical device for piercing bone has a cannula, a trocar positionable within the cannula, and an inner elongated pin positionable within the trocar. The actuation member is connected at an end of the inner elongated pin, wherein the inner elongated pin is deployable and retractable, wherein when in a deployed state, a tip of the inner elongated pin is positioned a greater distance out of a terminating end of the trocar than when in a retracted state, and wherein the inner elongated pin is deployable and retractable while positioned within the trocar.

The present disclosure can also be viewed as providing a system for piercing bone. Briefly described, in architecture, one embodiment of the system, among others, can be implemented as follows. A surgical system for piercing bone, comprising a cannula, a trocar positionable within the cannula, an inner elongated pin positionable within the trocar, wherein an actuation member is connected at an end of the inner elongated pin, wherein the inner elongated pin is deployable and retractable, wherein when in a deployed state, the inner elongated pin is configured to create an opening in a bone, an inner elongated pin tip configured to be placed in the opening to guide the trocar when the inner elongated pin is in a retracted state, wherein the trocar is configured to be advanced into the opening, and one or more surgical hardware configured to be inserted into the opening in the bone.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a partial cross-sectional illustration of a surgical device for piercing bone, in accordance with the present disclosure.

FIGS. 2A-2C are partial cross-sectional illustrations of the surgical device for piercing bone of FIG. 1 in a disassembled configuration, in accordance with the present disclosure.

FIG. 3A is a side plan view of the surgical device for piercing bone positioned over a bone, in accordance with the present disclosure.

FIG. 3B is a side plan view of the inner elongated pin of the surgical device for piercing bone in a deployed position inside of the bone, in accordance with the present disclosure.

FIG. 3C is a side plan view of the inner elongated pin of the surgical device for piercing bone in a retracted position and the trocar inside of the bone, in accordance with the present disclosure.

FIG. 4 is a detailed, cross-sectional side plan view of the surgical device for piercing the bone positioned over a bone, as depicted in FIG. 3A, in accordance with the present disclosure.

FIG. 5 is a detailed side plan view of the of the trocar inside of the bone with a support structure disposed thereon to provide a flush seal and support to the surgical device, in accordance with the present disclosure.

FIG. 6 is a flowchart illustrating a surgical method of piercing bone, in accordance with the present disclosure.

### DETAILED DESCRIPTION

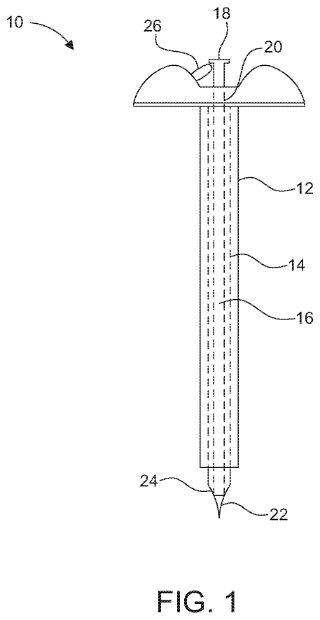

FIG. 1 is a partial cross-sectional illustration of a surgical trocar for piercing bone 10 (hereinafter “surgical device 10”), in accordance with the present disclosure. The surgical device 10 has a cannula 12, depicted in partial cross-section, and a trocar 14. The trocar 14 is positionable within the cannula 12. An inner elongated pin 16 is positionable within the trocar 14. An actuation member 18 is connected at an end 20 of the inner elongated pin 16. The inner elongated pin 16 is deployable and retractable. When in a deployed state, an inner elongated pin tip 22 is positioned a greater distance out of a trocar terminating end 24 than when in a retracted state. The inner elongated pin 16 is deployable and retractable while positioned within the trocar 14.

The cannula 12, trocar 14, and inner elongated pin 16 may be formed as separate components that are configured to interface with one another either directly or indirectly. When the surgical device 10 is initially assembled, the inner elongated pin 16 may be secured in the retracted position by the locking mechanism 26. The locking mechanism 26 may be a swivel lock or other protrusion that interfaces with the actuation member 18. The locking mechanism 26 prevents movement of the actuation member 18 by substantially securing it or locking it in an unengaged position. When in the unengaged position, the inner elongated pin tip 22 may protrude only partially from the trocar terminating end 24, thereby acting as the tip of the trocar 14.

The actuation member 18 may be a surface, structure, button, or similar device which may be pushed, pulled, or twisted by a user to deploy the inner elongated pin 16. The actuation member 18 may be physically or mechanically connected to the inner elongated pin 16, such as a blunt portion of the inner elongated pin 16 which is accessible proximate to the trocar head 34. In some examples, the inner elongated pin 16 may be deployed upon receiving a force from an object, such as a hammer or mallet 46, impacting the actuation member 18. The force may be an immediate force which causes quick movement of the inner elongated pin 16 relative to the cannula 12 or trocar 14. In other examples, the force may be a constant pressure force which is applied to the actuation member 18, and which is released instantaneously to cause movement of the inner elongated pin 16.

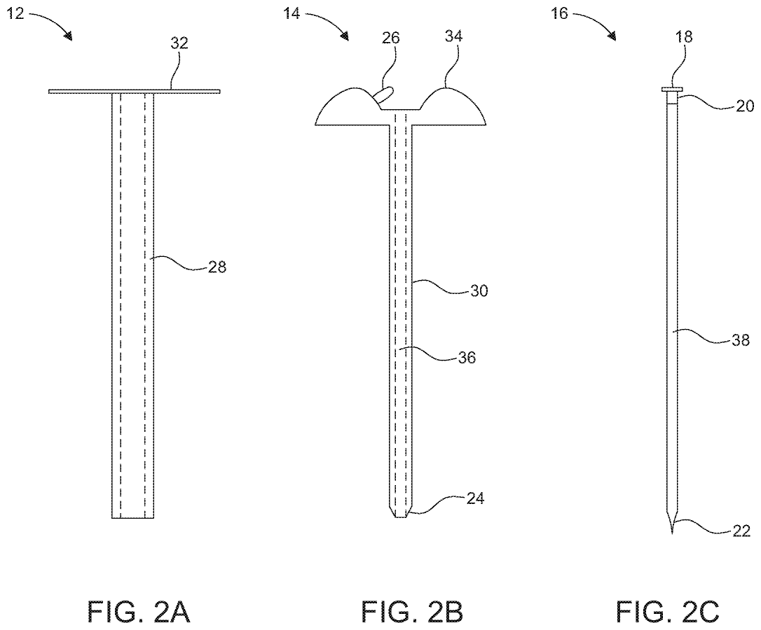

FIGS. 2A-2C are partial cross-sectional illustrations of the surgical device 10 of FIG. 1 in a disassembled configuration. With reference to FIGS. 2A-2C, FIG. 2A illustrates a cross-sectional view of a cannula 12. The cannula 12 has an internally housed trocar path 28 for receiving a portion of the length of the trocar 14 and trocar body 30. The cannula 12 may have a flange portion 32 configured to interface with the trocar head 34 when the trocar body 30 is removably positioned inside of the trocar path 28 of the cannula 12. The flange portion 32 and trocar head 34 may be reinforced with a durable material such as metal, plastic, or composite material to withstand the axial and normal forces arising from the force applied to the actuation member 18. The cannula 12 may be made of metal, plastic, composite, or any other material that is medical grade or surgical grade. The cannula 12 may have numeric labels disposed along the outer surface to allow a user to visualize or measure the depth of the cannula 12.

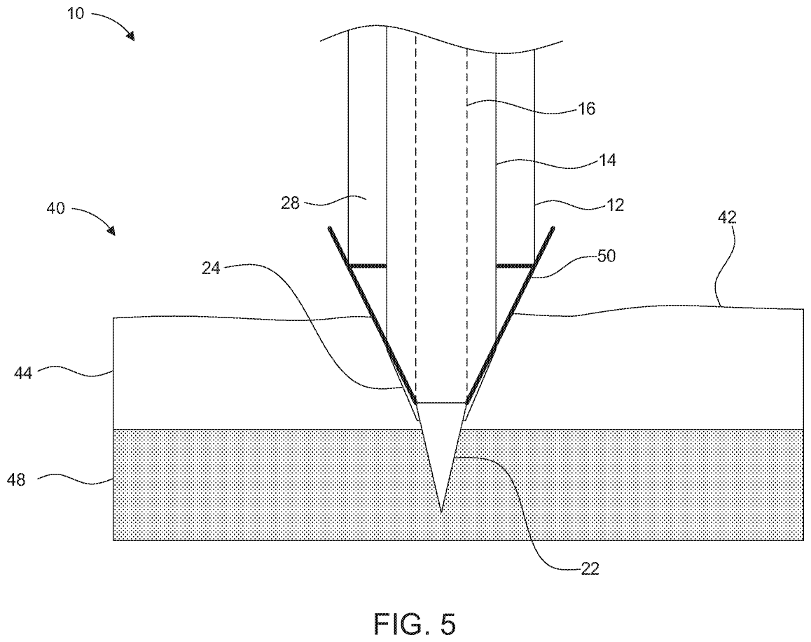

FIG. 2B illustrates a trocar 14 configured for insertion into a trocar path 28 of a cannula 12. The trocar body 30 may have the locking mechanism 26 positioned thereon. The locking mechanism 26 may be any locking mechanism 26 which substantially limits the movement of the inner elongated pin 16 when the locking mechanism 26 is in an engaged position. The locking mechanism 26 may be physically or mechanically connected to the trocar body 30 and may be disposed on an area proximal to actuation member 18. In one example, the locking mechanism 26 may be a swivel lock that is configured to interface with or contact a portion of the actuation member 18 in a manner that would substantially hinder movement of the actuation member 18. The trocar body 30 may have an inner elongated pin path 36 to receive at least a portion of the inner elongated pin 16 and inner elongated pin body 38. The inner elongated pin path 36 may extend into the trocar head 34 of the trocar 14 such that the actuation member 18 protrudes from the trocar head 34. A portion of the inner elongated pin body 38 may also partially protrude from the trocar head 34. The trocar terminating end 24 may be configured as a substantially blunt end having an opening to receive the inner elongated pin tip 22 and may substantially or entirely house the inner elongated pin tip 22. As discussed relative to FIG. 5, the trocar terminating end 24 may have a support structure 50 (FIG. 5) to removably couple the trocar 14 to the cannula 12. The support structure 50 (FIG. 5) may form a seal or substantially flush connection between the trocar terminating end 24, the cannula 12, and the inner elongated pin tip 22 in the unengaged position to prevent debris from entering the trocar 14. The trocar terminating end 24 may be angled, sloped, tapered, or otherwise configured to further pierce into the bone 40. The trocar 14 may be made of metal, plastic, composite, or any other material that is medical or surgical grade. The trocar 14 may be a partially or substantially sharpened trocar 14 or a blunt trocar 14 or obturator.

FIG. 2C illustrates the inner elongated pin 16 configured for insertion into the inner elongated pin path 36. The inner elongated pin 16 may have an actuation member 18 attached to an end 20, which may be configured as an extension of the inner elongated pin body 38 that can be manipulated, turned, pressed, pulled or otherwise acted upon by a user when the inner elongated pin 16 is positioned within the trocar 14. The inner elongated pin tip 22 may be configured as a tapered, sharpened, or substantially sharpened tip. The inner elongated pin tip 22 may also be configured to be threaded, ribbed, textured, have teeth, and/or the like to assist in bone removal, stability, durability, and piercing. The actuation member 18 may be a button or an extension of the inner elongated pin 16, and may be substantially flat, rounded, concave, or any other shape to allow for efficient actuation by a user. In an example where the actuation member 18 is an extension of the inner elongated pin 16 and the inner elongated pin 16 is inserted into inner elongated pin path 36 of the trocar 14, the actuation member 18 may be engaged by pressing or pushing, thereby causing the inner elongated pin 16 to move and protrude further outwards from the trocar terminating end 24. The inner elongated pin 16 may be made from metal, plastic, a composite material, or any other material which is medical or surgical grade or would otherwise provide durability.

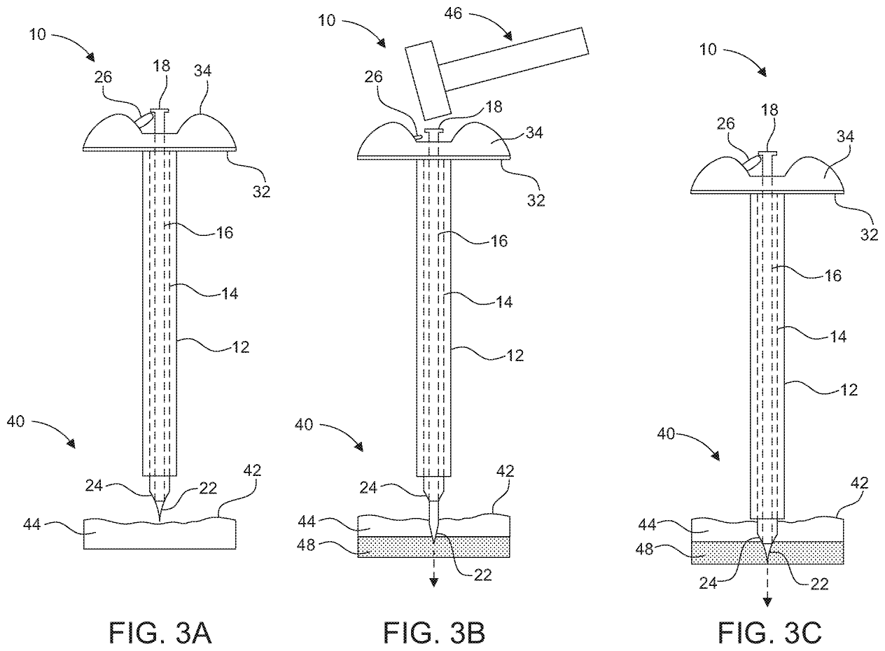

FIG. 3A is a side plan view of the surgical device 10 positioned over a bone 40. The surgical device 10 is in the fully assembled configuration when the trocar 14 is in position within the cannula 12 and the inner elongated pin 16 is positioned within the trocar 14. The actuation member 18 of the inner elongated pin 16 may be secured in a retracted position by the locking mechanism 26. In the retracted position, only a portion of the inner elongated pin body 38 (FIG. 2C) which forms the inner elongated pin tip 22 protrudes from the trocar terminating end 24. In one nonlimiting example where the inner elongated pin 16 is secured in a retracted position by the locking mechanism 26, the inner elongated pin 16 may not move relative to the inner elongated pin path 36 when an impact force is acted on it by way of a mallet 46 or hammer. The inner elongated pin tip 22 may be positioned over or directly in contact with the surface of the bone 40 by a user or surgeon at a desired location where a hole, opening, or aperture is desired. The bone 40 may be a vertebral pedicle, or any other bone within the human body. The inner elongated pin tip 22 may contact or interface with the periosteum 42, the outermost layer of cortical bone 44 when being positioned over the bone 40 prior to deploying the inner elongated pin 16. The inner elongated pin tip 22 may prevent displacement of the surgical device 10 when positioned over or directly in contact with the surface of the bone 40, since the inner elongated pin tip 22 may be easily lodged in the bone 40 with a quick movement, thereby limiting displacement of the surgical device 10. FIG. 3A includes many of the same features and structures as described relative to FIGS. 1-2C, which are not restated for brevity in disclosure.

FIG. 3B is a side plan view of the inner elongated pin 16 of the surgical device 10 in a deployed position inside of the bone 40. The inner elongated pin 16 may be deployed by removing, displacing, or moving the locking mechanism 26 such that it does not interface with or contact the actuation member 18 in a manner that would substantially hinder movement of the actuation member 18. In one example where the locking mechanism 26 is not removed, and therefore is still in a locking position, the trocar terminating end 24 may pierce or punch into the bone 40 simultaneously with the inner elongated pin tip 22. When the locking mechanism 26 is removed or unlocked, the inner elongated pin 16 may move freely or may have substantial resistance to only deploy upon a force acting upon the actuation member 18.

The actuation member 18 may be contacted with a mallet 46 or hammer to deploy the inner elongated pin 16. When deployed, the inner elongated pin body 38 (FIG. 2C) beyond the inner elongated pin tip 22 may extend out a predetermined distance and may pierce through the periosteum 42 and cortical bone 44. The trocar terminating end 24 may rest against the bone 40, remain at substantially the same vertical height, or move dynamically as the actuation member 18 is contacted with the mallet 46 or hammer. The predetermined distance may be varied or determined by arranging the locking mechanism 26 in different positions, such that the actuation member 18 may only be moved a certain distance before contacting the locking mechanism 26 and preventing further deployment of the inner elongated pin 16. In some examples, the locking mechanism 26 may be entirely removed or moved to allow the actuation member 18 to be fully engaged or pressed such that the inner elongated pin 16 is deployed a maximum travel distance. In some examples, the maximum travel distance may be approximately 1 centimeter.

The inner elongated pin 16 and inner elongated pin tip 22 may be configured and sharpened to punch, pierce, or drive through the cortical bone 44 or another bone 40 while providing stability to the user. The sharpened inner elongated pin tip 22 may stabilize the surgical device 10 as an impact force is acted on it by way of a mallet 46 or hammer. After the inner elongated pin 16 pierces through the cortical bone 44 and reaches, or substantially reaches trabecular bone 48, the inner elongated pin 16 may be placed back into the retracted state. The inner elongated pin 16 may be placed back in the retracted state by a user pulling back on the actuation member 18 and placing the locking mechanism 26 in contact with or to interface the actuation member 18. In some examples, the inner elongated pin 16 may be removed from the inner elongated pin path 36 entirely. When in the retracted state, the inner elongated pin tip 22 may at least partially protrude from the trocar terminating end 24, thereby acting as the tip of the trocar 14. For example, in the retracted state, the inner elongated pin tip 22 may be exposed while the inner elongated pin 16 is positioned inside the inner elongated pin path 36 of the trocar body 30. FIG. 3B includes many of the same features and structures as described relative to FIGS. 1-3A, which are not restated for brevity in disclosure.

FIG. 3C is a side plan view of the inner elongated pin 16 of the surgical device 10 in a retracted position and the trocar 14 inside of the bone 40. Once the inner elongated pin 16 is retracted, a hole or opening may be created through the bone 40, particularly through at least the cortical bone 44, and may extend at least partially into the trabecular bone 48. This hole or opening may serve as a guide or pilot hole for the remainder of the trocar 14 to be inserted thereto. Since the hole is sized to receive the inner elongated pin 16, once the inner elongated pin 16 is retracted, the inner elongated pin tip 22 may be placed in the hole or opening in the bone 40. Once placed, the trocar 14 may be advanced to create a wider and deeper hole or opening in the bone 40. The trocar terminating end 24 may be angled or sloped to further pierce or punch into the bone 40. The inner elongated pin tip 22 may continue to guide the trocar 14 through the trabecular bone 48 until a desired depth is reached. Once the desired depth into the bone 40 is reached, the surgical device 10 may be partially or entirely removed from the bone 40. The surgical device 10 can be repositioned over or on the surface of the bone 40 for repeated deployment of the inner elongated pin 16. An opening or hole, substantially having the same diameter as the trocar 14, may be formed in the bone 40, through the cortical bone 44 and into the trabecular bone 48. This hole or opening may be configured to receive surgical hardware. In an example where the bone 40 is a vertebral pedicle, pedicle screws may be inserted into the bone 40.

The use of an inner elongated pin 16 to be deployed and create an initial pilot hole for the remainder of the trocar 14 provides ease to a user or surgeon in creating holes large enough to receive surgical hardware such as one or more screws, screwdrivers, and/or guide wires. Driving a trocar 14 without the assistance of a guide or pilot hole may be difficult when working on uneven bone 40 surfaces such as vertebrae or vertebral pedicles. The thickness of a trocar 14 typically results in slippage when the trocar head 34 is struck with a hammer. The use of a pilot hole formed by an inner elongated pin 16 provides initial stability to drive the surgical device 10 through the periosteum 42 and cortical bone 44. Incorporating the inner elongated pin tip 22 as a continuation or as a tip of the trocar 14 continually provides stability as the trocar 14 is advanced to widen the hole or opening of the cortical bone 44 and advance through the trabecular bone 48. FIG. 3C includes many of the same features and structures as described relative to FIGS. 1-3B, which are not restated for brevity in disclosure.

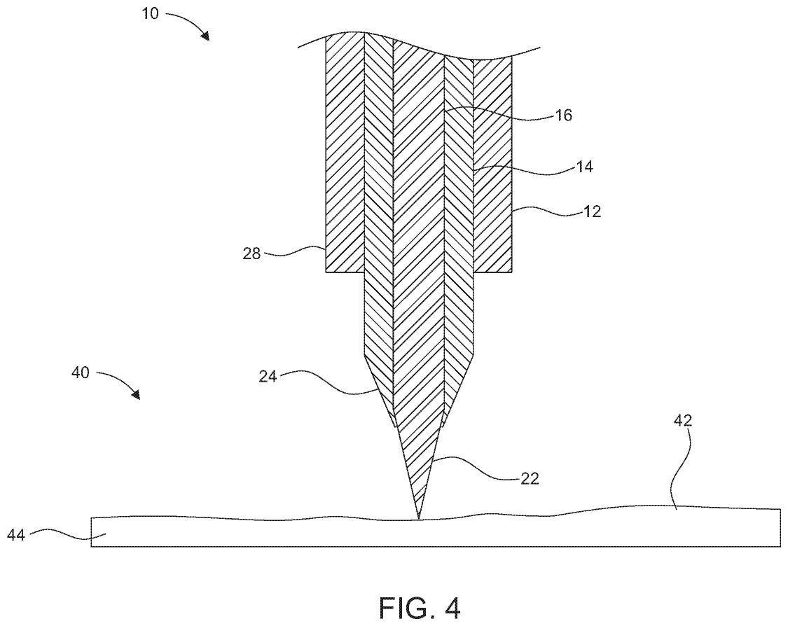

FIG. 4 is a detailed, partial cross-sectional view of the surgical device 10 for piercing the bone positioned over a bone 40. The surgical device 10 is in the fully assembled configuration when the trocar 14 is in position within the cannula 12 and the inner elongated pin 16 is positioned within the trocar 14. FIG. 4 includes many of the same features as described earlier herein, which are not repeated for brevity of the disclosure.

FIG. 5 is a detailed illustration of the of the surgical device 10 with a support structure 50 disposed thereon to provide a flush seal and support to the surgical device 10 in a position against the bone 40. The trocar terminating end 24 may be reinforced with the support structure 50 to withstand contact with the bone 40 when the actuation member 18 is contacted with the mallet 46 or hammer. The support structure 50 may be physically or mechanically connected to the trocar terminating end 24. The support structure 50 may be substantially flat or rounded and may be reinforced with a durable material such as metal, plastic, composite, or any other material that is medical grade or surgical grade. In the example shown in FIG. 5, the support structure 50 may be conical shaped such that a portion of the support structure 50 extends past the outer surface of the bone 40 while another portion is positioned inset within the bone 40. The support structure 50 may prevent displacement of the surgical device 10 and withstand tangential and axial forces against the trocar terminating end 24 during placement and/or insertion of the inner elongated tip 22. The support structure 50 may be positioned as to not impede the inner elongated pin 16 from retracting and/or deploying.

As discussed earlier herein, the inner elongated pin tip 22 may be substantially flush or otherwise form a seal with the trocar terminating end 24 in the unengaged position to prevent debris from entering the trocar 14. As illustrated in FIG. 5, in the unengaged position, the support structure 50 may form a flush seal between the trocar terminating end 24, cannula 12, and inner elongated pin tip 22. In some examples, there may be a flush seal formed between the cannula 12, trocar terminating end 24, and inner elongated pin tip 22 in the engaged position. The seal may be formed from close contact between the cannula 12, trocar terminating end 24, and inner elongated pin tip 22, or by a medical or surgical material that forms a seal such as rubber, plastic, metal, and/or the like.

With reference to FIGS. 1-4, various methods of using the surgical device 10 are contemplated. One example of a method may be summarized as follows. Using a surgical device 10 for piercing bone 40 includes steps of positioning the trocar 14 within the cannula 12. An inner elongated pin 16 is positioned within the trocar 14, wherein the inner elongated pin 16 is deployable and retractable. The inner elongated pin tip 22 may be positioned over or on a bone 40 surface or periosteum 42. The actuation member 18 may be actuated by a force, such as from a mallet 46 or hammer. Actuating the actuation member 18 deploys the inner elongated pin 16 to pierce through at least a portion of cortical bone 44. When in the deployed state, the inner elongated pin tip 22 may be emerged a greater distance out of the trocar terminating end 24 than when the inner elongated pin tip 22 is in the retracted state. The actuation member 18 may then be retracted, thereby positioning the inner elongated pin 16 in the retracted mode. The actuation member 18 may be locked in a substantially constrained position by the locking mechanism 26. The trocar terminating end 24 and at least a portion of the trocar body 30 may then be driven through the cortical bone 44 and into at least a portion of trabecular bone 48. After creating a hole or opening within the bone 40, the surgical device 10 may be removed. Surgical hardware, such as screws, screwdrivers, pins, rods, guide wires, and the like, may be positioned within the hole or opening created by the surgical device 10.

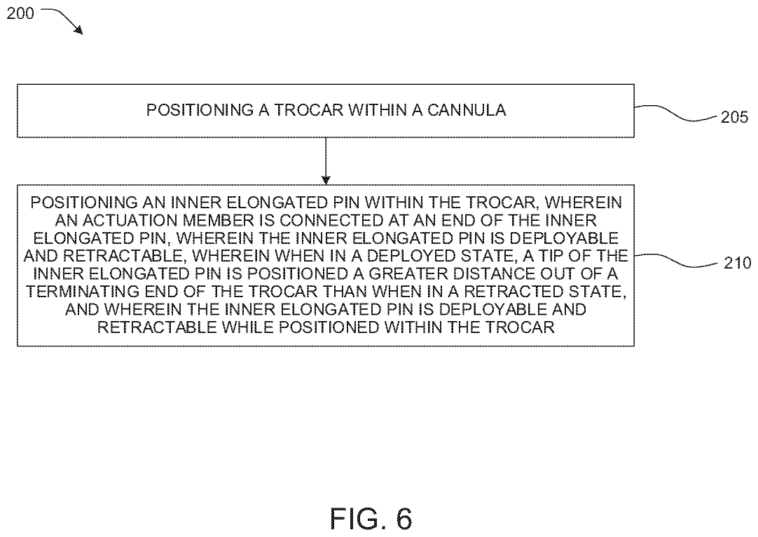

FIG. 6 is a flowchart 200 illustrating a method of piercing bone in accordance with embodiments of the disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by step 205, a trocar is positioned within a cannula. At step 210, the inner elongated pin is positioned within the trocar, wherein the inner elongated pin is deployable and retractable. When in a deployed state, a tip of the inner elongated pin is positioned a greater distance out of a terminating end of the trocar than when in a retracted state, and the inner elongated pin is deployable and retractable while positioned within the trocar.

In furtherance of the described method, and with reference to FIGS. 1-6, in order to restrict unwanted movement of the inner elongated pin 16, the trocar 14 may comprise a locking mechanism 26 configured to prevent movement of the actuation member 18. The inner elongated pin 16 may be deployed via a displacement of the locking mechanism 26, and/or may be deployed via a force actuating the actuation member 18. The inner elongated pin tip 22 may be configured to pierce through a cortical bone 44. When in the retracted state, the tip of the inner elongated pin 22 may partially, substantially, or minimally protrude from the terminating end of the trocar 24. The distance that the inner elongated pin 16 may extend in the deployed state may be a predetermined distance variable by a position of the locking mechanism 26. Additionally, a method 200 to use the surgical device 10 may include the process of a user applying a force to actuate the actuation member 18, which therefore may deploy the inner elongated pin 16 to create an opening in a bone 40. Once the user has deployed the inner elongated pin 16, the inner elongated pin 16 may then be retracted to the retracted state. The inner elongated pin tip 22 may then be placed in the opening of the bone 40. Once the inner elongated pin tip 22 is placed in the opening, the trocar 14 may be advanced towards and/or into the bone 40 to create a wider opening. Any number of additional steps, functions, processes, or variants thereof may be included in the method, including any disclosed relative to any other figure of this disclosure.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. A surgical device for piercing bone, comprising;
a cannula;
a trocar positionable within the cannula; and
an inner elongated pin positionable within the trocar, wherein an actuation member is connected at an end of the inner elongated pin, wherein the inner elongated pin is deployable and retractable, wherein when in a deployed state, a tip of the inner elongated pin is positioned a greater distance out of a terminating end of the trocar than when in a retracted state, and wherein the inner elongated pin is deployable and retractable while positioned within the trocar.

2. The surgical device of claim 1, wherein the trocar comprises a locking mechanism configured to prevent movement of the actuation member.

3. The surgical device of claim 2, wherein the inner elongated pin is deployed via a displacement of the locking mechanism.

4. The surgical device of claim 1, wherein the inner elongated pin is deployed via a force actuating the actuation member.

5. The surgical device of claim 1, wherein in the retracted state, the tip of the inner elongated pin partially protrudes from the terminating end of the trocar.

6. The surgical device of claim 1, wherein the distance out of the terminating end of the trocar is a predetermined distance variable by a position of the locking mechanism.

7. The surgical device of claim 1, wherein the inner elongated pin tip is configured to pierce through a cortical bone.

8. The surgical device of claim 1, wherein the trocar comprises a locking mechanism configured to prevent movement of the actuation member.

9. The surgical device of claim 1, wherein the one or more surgical hardware comprises at least one of a guide wire, screwdriver, one or more screws, or one or more rods.

10. A surgical method for piercing bone, comprising:
positioning a trocar within a cannula; and
positioning an inner elongated pin within the trocar, wherein an actuation member is connected at an end of the inner elongated pin, wherein the inner elongated pin is deployable and retractable, wherein when in a deployed state, a tip of the inner elongated pin is positioned a greater distance out of a terminating end of the trocar than when in a retracted state, and wherein the inner elongated pin is deployable and retractable while positioned within the trocar.

11. The method of claim 10, further comprising:
actuating the actuation member via a force, thereby deploying the inner elongated pin to create an opening in a bone;
retracting the inner elongated pin;
placing the inner elongated pin tip in the opening; and
advancing the trocar to create a wider opening.

12. The surgical method of claim 10, wherein the trocar comprises a locking mechanism configured to prevent movement of the actuation member.

13. The surgical method of claim 12, wherein the inner elongated pin is deployed via a displacement of the locking mechanism.

14. The surgical method of claim 10, wherein the inner elongated pin is deployed via a force actuating the actuation member.

15. The surgical method of claim 10, wherein in the retracted state, the tip of the inner elongated pin partially protrudes from the terminating end of the trocar.

16. The surgical method of claim 10, wherein the distance out of the terminating end of the trocar is a predetermined distance variable by a position of the locking mechanism.

17. The surgical method of claim 10, wherein the inner elongated pin tip is configured to pierce through a cortical bone.

18. A surgical system for piercing bone, comprising:
a cannula;
a trocar positionable within the cannula;
an inner elongated pin positionable within the trocar, wherein an actuation member is connected at an end of the inner elongated pin, wherein the inner elongated pin is deployable and retractable, wherein when in a deployed state, the inner elongated pin is configured to create an opening in a bone;
an inner elongated pin tip configured to be placed in the opening to guide the trocar when the inner elongated pin is in a retracted state, wherein the trocar is configured to be advanced into the opening; and
one or more surgical hardware configured to be inserted into the opening in the bone.

19. The surgical system of claim 18, wherein the inner elongated pin tip is configured to pierce through a cortical bone.

20. The surgical system of claim 18, wherein the inner elongated pin tip is further configured to be threaded.
